(19) United States
(12) Patent Application Publication
(10) Pub. No.: US 2024/0198004 A1
(43) Pub. Date: Jun. 20, 2024

(54) NEEDLELESS SYRINGE

(71) Applicant: BAZ BIOMEDIC CO., LTD., Seoul (KR)

(72) Inventors: Sung Hun LEE, Gyeonggi-do (KR); Jung Kook KIM, Daejeon (KR); Hwi HAM, Seoul (KR)

(73) Assignee: BAZ BIOMEDIC CO., LTD., Seoul (KR)

(21) Appl. No.: 18/287,645

(22) PCT Filed: Mar. 4, 2021
(86) PCT No.: PCT/KR2021/002694

§ 371 Date: Oct. 19, 2023

(30) Foreign Application Priority Data
Mar. 6, 2020 (KR) 10-2020-0028432

(51) Int. Cl. A61M 5/30

(52) U.S. Cl. CPC A61M 5/30; A61M 2205/8287

(57) ABSTRACT

A needleless syringe according to the present invention including a moving magnetic body and a stationary magnet wherein, when a piston moves forward, a magnetic field is generated in a direction opposite to the magnetic field direction of the stationary magnet to offset the magnetic field of the stationary magnet and thus separating the moving magnetic body and the stationary magnet from each other and, when the piston moves backward, the moving magnetic body is attracted using the magnetic field of the stationary magnet so that the piston for pressurizing and injecting a drug can repeatedly and reciprocally move forward or backward, thereby rapidly and repeatedly injecting a predetermined drug.

### TECHNICAL FIELD

The present invention relates to a needleless syringe, and more particularly, to a needleless syringe capable of repeatedly injecting a drug at high speed without an injection needle.

### BACKGROUND ART

In general, a syringe is a device for injecting a medicinal solution into the tissue of an organism. The syringe includes a needle inserted into the body, a syringe cylinder in which the medicinal solution is accommodated, and a piston that reciprocates inside the syringe cylinder and pushes the medicinal solution with the needle. The needle is punctured to allow a drug to be injected when injected.

Recently, in order to relieve the fear of the needle of the syringe and to prevent infection due to the needle, research and development on the syringe without the needle has been actively carried out.

However, because the existing needleless syringe is configured to inject a predetermined amount of a drug into only one part of the skin at a time, damage to the skin tissue may occur.

In addition, because discomfort such as reloading after one injection follows, there is a limitation in that the needleless syringe cannot be used to evenly inject the drug multiple times into a large area of the skin in the field of skin care and the like.

### DETAILED DESCRIPTION OF THE INVENTION

### Technical Problem

The objective of the present invention is to provide a needleless syringe in which a small amount of a drug is repeatedly injected at high speed and is evenly injected into a larger area of the skin.

### Technical Solution

According to an aspect of the present invention, there is provided a needleless syringe including: a body formed in a hollow shape; a solenoid coil wound on an outer circumferential surface of the body; a cylinder coupled to the body to be in communication with an open front surface of the body and including a drug accommodating portion in which the drug is accommodated, and a nozzle portion injecting the drug accommodated in the drug accommodating portion forward; a moving magnetic body that is long inserted into the body in a longitudinal direction and reciprocates forward and backward by a magnetic force generated when a current is applied to the solenoid coil; a stationary magnet that is inserted and fixed behind the moving magnetic body inside the body; a piston that is inserted in front of the moving magnetic body inside the body, moves forward by an impulse applied by the moving magnetic body when the moving magnetic body moves forward, to pressurize the drug in the drug accommodating portion toward the nozzle portion; a nozzle portion opening/closing valve that is provided to open/close a passage hole between the nozzle portion and the drug accommodating portion, is pushed by a fluidic pressure applied by the drug from the drug accommodating portion during forward movement of the piston to open the passage hole, and is elastically restored when the fluidic pressure is released to close the passage hole; and a forward/backward driving unit that repeats the supply and cut-off of current to the solenoid coil at a preset period, thereby repeating the forward movement and the backward movement of the piston, wherein the solenoid coil includes: a first coil, which is wound on front of the outer circumferential surface of the body, to which a current is applied during forward movement of the piston and generates a magnetic force in a forward movement direction of the piston; and a second coil, which is wound on rear of the first coil on the outer circumferential surface of the body, to which a current is applied during forward movement of the piston and which generates a magnetic force in an opposite direction to a direction of a magnetic field of the stationary magnet to offset the magnetic field of the stationary magnet and in which supply of current is cut off when the piston moves backward, to restore the magnetic field of the stationary magnet, and the forward/backward driving unit includes: a current supply unit that repeatedly supplies a current to each of the first coil and the second coil during forward movement of the piston to move the moving magnetic body forward and repeatedly cuts off supply of current to each of the first coil and the second coil during backward movement of the piston to move the moving magnetic body backward due to the magnetic field of the stationary magnet; and an elastic member that is installed between the piston and the cylinder and applies an elastic force in a backward movement direction to the piston when the moving magnetic body moves backward.

The current supply unit may supply a current to the first coil for a first preset time and then cuts off supply of current, and a mass of the moving magnetic body may be 100 g or less, and the first preset time may be set to be 180 ms or less.

The current supply unit may apply a current to the second coil and then, when a second preset time elapses, the current supply unit may apply a current to the first coil, and when the piston moves backward, the current supply unit may cut off supply of current to the first coil and then, when a third preset time elapses, the current supply unit may cut off supply of current to the second coil.

According to another aspect of the present invention, there is provided a needleless syringe including: a body formed in a hollow shape; a solenoid coil wound on an outer circumferential surface of the body; a cylinder coupled to the body to be in communication with an open front surface of the body and including a drug accommodating portion in which the drug is accommodated, and a nozzle portion injecting the drug accommodated in the drug accommodating portion forward; a moving magnetic body that is long inserted into the body in a longitudinal direction and reciprocates forward and backward by a magnetic force generated when a current is applied to the solenoid coil; a stationary magnetic body that is inserted behind the moving magnetic body inside the body and has magnetism by a magnetic force generated when a current is applied to the solenoid coil and is fixed; a piston that is inserted in front of the moving magnetic body inside the body, moves forward by an impulse applied by the moving magnetic body when the moving magnetic body moves forward, to pressurize the drug in the drug accommodating portion toward the nozzle portion; a nozzle portion opening/closing valve that is provided to open/close a passage hole between the nozzle portion and the drug accommodating portion, is pushed by a fluidic pressure applied by the drug from the drug accommodating portion during forward movement of the piston to open the passage hole, and is elastically restored when the fluidic pressure is released to close the passage hole; and a forward/backward driving unit that repeats the supply and cut-off of current to the solenoid coil at a preset period, thereby repeating the forward movement and the backward movement of the piston, wherein the solenoid coil includes: a first coil, which is wound on front of the outer circumferential surface of the body, to which a current is applied during forward movement of the piston and generates a magnetic force in a forward movement direction of the moving magnetic body; and a second coil, which is wound on rear of the first coil on the outer circumferential surface of the body, to which a current is applied during backward movement of the piston and which generates a magnetic force to magnetize the stationary magnetic body, and the forward/backward driving unit includes: a current supply unit that repeatedly supplies a current to the first coil to move the moving magnetic body forward and cuts off supply of current to the second coil during forward movement of the piston and that repeatedly cuts off supply of current to the first coil and supplies a current to the second coil to move the moving magnetic body backward due to the magnetic field of the stationary magnetic body during backward movement of the piston; and an elastic member that is installed between the piston and the cylinder and applies an elastic force in a backward movement direction to the piston when the moving magnetic body moves backward, and the current supply unit applies a current to the first coil for a first preset time and then cuts off supply of current, and a mass of the moving magnetic body is 100 g or less, and the first preset time is set to be 80 ms or less.

The needleless syringe may further include a cooling chamber that is provided to surround an outside of the solenoid coil on an outside of the body and absorbs and cools heat generated in the solenoid coil through a cooling fluid.

The needleless syringe may further include a flange portion that protrudes in a radial direction and is formed on an outer circumferential surface of the piston and a blocker that is provided between the piston and the cylinder and limits a forward movement distance of the piston due to the flange portion blocked when the piston moves forward, wherein the blocker includes a fixed blocker that is fixedly installed to an inner circumferential surface of the cylinder and has a female thread formed on an inner circumferential surface of the fixed blocker and formed in a ring shape, and a length adjustment blocker that is screwed to an inner circumferential surface of the fixed blocker, protrudes backward so that the flange portion is blocked by the length adjustment blocker, adjusts a coupling length at which the length adjustment blocker is coupled to the fixed blocker.

The drug accommodating portion may be formed in a shape of a diverging nozzle including a reduced portion whose cross-sectional area gradually decreases toward the front, and an enlarged portion that extends from the reduced portion and increases in cross-sectional area again, and a drug supply hole for supplying the drug from the outside by a pressure difference generated when the piston moves backward, may be formed in the reduced portion.

The nozzle portion opening/closing valve may include a ball that is installed in the passage hole and an elastic member that is installed in the nozzle portion and supports the ball.

The current supply unit may include a capacitor that is connected to the first coil, stores the current supplied from an external power supply source, supplies the stored current to the first coil when the piston moves forward, and cuts off supply of power to the first coil when the piston moves backward, and a direct current (DC) power supply unit that is connected to the second coil and supplies the current supplied from the external power supply source to the second coil when the piston moves forward.

The needleless syringe may further include a piston cover that is provided inside the cylinder to cover an end portion of the piston and is formed of a stretchable material so as to be stretched by the piston when the piston moves forward or backward.

According to another aspect of the present invention, there is provided a needleless syringe including: a body formed in a hollow shape; a solenoid coil wound on an outer circumferential surface of the body; a cylinder coupled to the body to be in communication with an open front surface of the body and including a drug accommodating portion in which the drug is accommodated, and a nozzle portion injecting the drug accommodated in the drug accommodating portion forward; a moving magnetic body that is long inserted into the body in a longitudinal direction and reciprocates forward and backward by a magnetic force generated when a current is applied to the solenoid coil; a stationary magnet that is inserted and fixed behind the moving magnetic body inside the body; a piston that is inserted in front of the moving magnetic body inside the body, moves forward by an impulse applied by the moving magnetic body when the moving magnetic body moves forward, to pressurize the drug in the drug accommodating portion toward the nozzle portion; a nozzle portion opening/closing valve that is provided to open/close a passage hole between the nozzle portion and the drug accommodating portion, is pushed by a fluidic pressure applied by the drug from the drug accommodating portion during forward movement of the piston to open the passage hole, and is elastically restored when the fluidic pressure is released to close the passage hole; and a forward/backward driving unit that repeats the supply and cut-off of current to the solenoid coil at a preset period, thereby repeating the forward movement and the backward movement of the piston, wherein the solenoid coil includes: a first coil, which is wound on front of the outer circumferential surface of the body, to which a current is applied during forward movement of the piston and generates a magnetic force in a forward movement direction of the piston; and a second coil, which is wound on rear of the first coil on the outer circumferential surface of the body, to which a current is applied during forward movement of the piston and which generates a magnetic force in an opposite direction to a direction of a magnetic field of the stationary magnet to offset the magnetic field of the stationary magnet and in which supply of current is cut off when the piston moves backward, to restore the magnetic field of the stationary magnet, and the forward/backward driving unit includes: a current supply unit that repeatedly supplies a current to each of the first coil and the second coil during forward movement of the piston to move the moving magnetic body forward and repeatedly cuts off supply of current to each of the first coil and the second coil to move the moving magnetic body backward due to the magnetic field of the stationary magnet; an elastic member that is installed between the piston and the cylinder and applies an elastic force in a backward movement direction to the piston when the moving magnetic body moves backward; and a piston cover that is provided inside the cylinder to cover an end portion of the piston and is formed of a stretchable material so as to be stretched by the piston when the piston moves forward or backward, and the current supply unit may apply a current to the first coil for a first preset time and then may cut off supply of current, and a mass of the moving magnetic body may be 100 g or less, and the first preset time may be set to be 180 ms or less, and the current supply unit may apply a current to the second coil and then, when a second preset time elapses, the current supply unit may apply a current to the first coil, and when the piston moves backward, the current supply unit may cut off supply of current to the first coil and then, when a third preset time elapses, the current supply unit may cut off supply of current to the second coil.

According to another aspect of the present invention, there is provided a needleless syringe including: a body formed in a hollow shape; a solenoid coil wound on an outer circumferential surface of the body; a cylinder coupled to the body to be in communication with an open front surface of the body and including a drug accommodating portion in which the drug is accommodated, and a nozzle portion injecting the drug accommodated in the drug accommodating portion forward; a moving magnetic body that is long inserted into the body in a longitudinal direction and moves forward by a first magnetic force generated when a current is applied to the solenoid coil; a stationary magnet that is inserted behind the moving magnetic body inside the body and has a second magnetic force to pull the moving magnetic body when supply of current to the solenoid coil is cut off, to move the moving magnetic body backward; a piston that is inserted in front of the moving magnetic body inside the body, moves forward by an impulse applied by the moving magnetic body when the moving magnetic body moves forward, to pressurize the drug in the drug accommodating portion toward the nozzle portion; a nozzle portion opening/closing valve that is provided to open/close a passage hole between the nozzle portion and the drug accommodating portion, is pushed by a fluidic pressure applied by the drug from the drug accommodating portion during forward movement of the piston to open the passage hole, and is elastically restored when the fluidic pressure is released to close the passage hole; and a forward/backward driving unit that repeats the supply and cut-off of current to the solenoid coil at a preset period, thereby repeating the forward movement and the backward movement of the piston, wherein the forward/backward driving unit includes: a current supply unit that repeatedly supplies a current to the solenoid coil to generate the first magnetic force greater than the second magnetic force during forward movement of the piston and repeatedly cuts off supply of current to the solenoid coil during backward movement of the piston; and an elastic member that is installed between the piston and the cylinder and applies an elastic force in a backward movement direction to the piston when the moving magnetic body moves backward.

According to another aspect of the present invention, there is provided a needleless syringe including: a body formed in a hollow shape; a solenoid coil wound on an outer circumferential surface of the body; a cylinder coupled to the body to be in communication with an open front surface of the body and including a drug accommodating portion in which the drug is accommodated, and a nozzle portion injecting the drug accommodated in the drug accommodating portion forward; a moving magnetic body that is long inserted into the body in a longitudinal direction and moves forward by a first magnetic force generated when a current in a preset first direction is applied to the solenoid coil; a stationary magnet that is inserted behind the moving magnetic body inside the body and has a second magnetic force to pull the moving magnetic body when supply of current to the solenoid coil is cut off, to move the moving magnetic body backward; a piston that is inserted in front of the moving magnetic body inside the body, moves forward by an impulse applied by the moving magnetic body when the moving magnetic body moves forward, to pressurize the drug in the drug accommodating portion toward the nozzle portion; a nozzle portion opening/closing valve that is provided to open/close a passage hole between the nozzle portion and the drug accommodating portion, is pushed by a fluidic pressure applied by the drug from the drug accommodating portion during forward movement of the piston to open the passage hole, and is elastically restored when the fluidic pressure is released to close the passage hole; and a forward/backward driving unit that repeats the supply and cut-off of current to the solenoid coil at a preset period, thereby repeating the forward movement and the backward movement of the piston, wherein the forward/backward driving unit includes: a current supply unit that repeatedly supplies the current in the first direction to the solenoid coil to generate the first magnetic force greater than the second magnetic force during forward movement of the piston and repeatedly supplies a current in a second direction opposite to the first direction to the solenoid coil during backward movement of the piston; and an elastic member that is installed between the piston and the cylinder and applies an elastic force in a backward movement direction to the piston when the moving magnetic body moves backward.

The needleless syringe may further include a cooling chamber that is provided to surround an outside of the solenoid coil on an outside of the body and absorbs and cools heat generated in the solenoid coil through a cooling fluid.

The needleless syringe may further include a piston cover that is provided inside the cylinder to cover an end portion of the piston and is formed of a stretchable material so as to be stretched by the piston when the piston moves forward or backward.

### Effects of the Invention

A needleless syringe according to the present invention is configured to include
a moving magnetic body and a stationary magnet wherein, when a piston moves forward, a magnetic field is generated in a direction opposite to the magnetic field direction of the stationary magnet to offset the magnetic field of the stationary magnet and thus separating the moving magnetic body and the stationary magnet from each other and, when the piston moves backward, the moving magnetic body is attracted using the magnetic field of the stationary magnet so that the piston for pressurizing and injecting a drug can repeatedly and reciprocally move forward or backward, thereby rapidly and repeatedly injecting a predetermined drug.

Moreover, the present invention minimizes, through controlling a time of applying current to a first coil, recoil without need to add a separate recoil offset structure thereto, and thus can improve convenience in use while having a simple structure.

In addition, a moving magnetic body collides with a piston to move the piston forward so that the drug can be injected at higher speed by using a smaller energy.

In addition, multiple injections, rather than a single injection, are possible during a single procedure to a wider range of skin such as that of the face in the fields of skin care and the like.

Furthermore, a user can automatically and repeatedly inject a small amount of drug at high speed without separate loading.

Furthermore, voltage applied to a solenoid coil and the coupling length of a length adjustment blocker are changed so that one injection amount of the drug can be adjusted.

Furthermore, a cooling chamber is provided around the solenoid coil so that a magnetic force can be prevented from being weakened by heat generated in the solenoid coil.

Furthermore, a piston cover is provided between a cylinder and the piston so as to prevent the drug from staining the end portion of the piston, and thus the user does not have to wipe the end portion of the piston.

### DESCRIPTION OF THE DRAWINGS

FIG. 1 is a longitudinal perspective view showing a needleless syringe according to a first embodiment of the present invention.

FIG. 2 is a view showing a forward movement state of a piston of the needleless syringe according to the first embodiment of the present invention.

FIG. 3 is a view showing a backward movement state of the piston of the needleless syringe according to the first embodiment of the present invention.

FIG. 4 is a graph showing a nozzle opening/closing valve of the needleless syringe according to the first embodiment of the present invention.

FIG. 5 is a graph showing a current supply waveform applied to each of a first coil and a second coil of the needleless syringe according to the first embodiment of the present invention.

FIG. 6 is a graph showing comparison of a recoil force (Force) according to time at which a current is applied to the first coil of the needless syringe according to the first embodiment of the present invention.

FIG. 7 is a graph showing a recoil distance (Displacement) according to time at which a current is applied to the first coil of the needless syringe according to the first embodiment of the present invention.

FIG. 8 is a longitudinal perspective view showing a needleless syringe according to a second embodiment of the present invention.

FIG. 9 is a graph showing a current supply waveform applied to each of a first coil and a second coil of the needleless syringe according to the second embodiment of the present invention.

FIG. 10 is a graph showing a recoil distance (Displacement) according to time at which a current is applied to the first coil of the needless syringe according to the second embodiment of the present invention.

FIG. 11 is a longitudinal cross-sectional view showing a configuration in which a cooling chamber is provided in a needless syringe according to a third embodiment of the present invention.

FIG. 12 is a longitudinal cross-sectional view showing a configuration in which a piston cover is provided in a needless syringe according to a fourth embodiment of the present invention.

FIG. 13 is a view showing a forward movement state of a piston of the needleless syringe according to a fifth embodiment of the present invention.

FIG. 14 is a view showing a backward movement state of the piston of the needleless syringe according to the fifth embodiment of the present invention.

FIG. 15 is a graph showing another example of a current supply waveform applied to a solenoid coil of a needleless syringe according to the fifth embodiment of the present invention.

FIG. 16 is a graph showing another example of a current supply waveform applied to a solenoid coil of a needleless syringe according to a sixth embodiment of the present invention.

### MODE OF THE INVENTION

Hereinafter, embodiments of the present invention will be described with reference to the accompanying drawings.

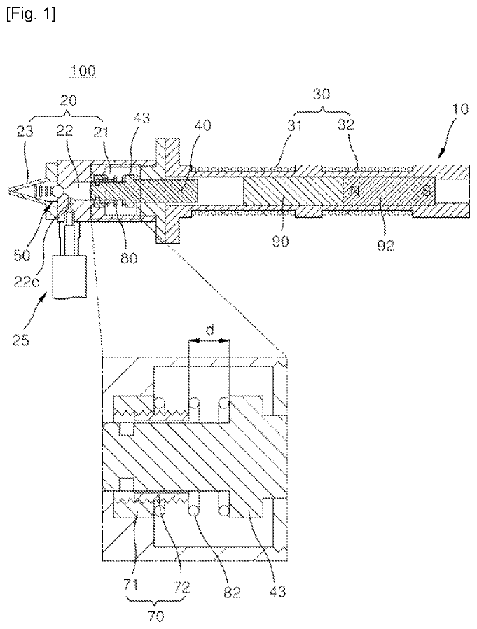

FIG. 1 is a longitudinal cross-sectional view showing a needleless syringe according to a first embodiment of the present invention.

Referring to FIG. 1, a needleless syringe 100 according to the first embodiment of the present invention includes a body 10, a cylinder 20, a solenoid coil 30, a moving magnetic body 90, a stationary magnet 92, a piston 40, a nozzle portion opening/closing valve 50, a forward/backward driving unit 60, a blocker 70, and an elastic member 80.

The needleless syringe 100 is an impact type syringe in which, when the moving magnetic body 90 moves forward by the magnetic force generated by the solenoid coil 30, the moving magnetic body 90 collides with the piston 40 to move the piston 40 forward.

The body 10 is formed in a hollow shape and is formed to be elongated in a longitudinal direction. The front surface of the body 10 is formed to be opened.

The cylinder 20 is screwed to the front of the body 10. The cylinder 20 is coupled to the body 10 to be in communication with the open front surface of the body 10.

The cylinder 20 is formed in a hollow shape and is formed in such a way that a cylinder main hole 21, a drug accommodating portion 22 and a nozzle portion 23 are in communication with each other.

The cylinder main hole 21 is formed in the rear of an inner side of the cylinder 20, and a thread is formed in such a way that the front end of the body 10 is inserted into at least a part of the cylinder 20 to be screw-coupled to each other.

The drug accommodating portion 22 is formed to have a reduced cross-sectional area compared to a body coupling hole 21. The drug accommodating portion 22 is a passage through which the piston 40 is in close contact with the drug accommodating portion 22, and is an accommodation space in which a drug is accommodated.

The drug accommodating portion 22 is formed in a shape of a diverging nozzle including a reduced portion 22a whose cross-sectional area gradually decreases toward the front, and an enlarged portion 22b that extends from the reduced portion 22a and increases in cross-sectional area again. A drug supply hole 22c for supplying the drug from the outside by a pressure difference generated when the piston 40 moves backward is formed in the reduced portion 22a. A drug filling device 25 is coupled to the drug supply hole 22c.

The nozzle portion 23 is formed to be in communication with the drug accommodating portion 22 and to have a gradually decreasing cross-sectional area, and injects the drug accommodated in the drug accommodating portion 22.

In the present embodiment, an example in which the cylinder 20 is formed by coupling a first block in which the body coupling hole 21 and the drug accommodating portion 22 are formed and a second block in which the nozzle portion 23 is formed, will be described. However, the present invention is not limited thereto, and the first block and the second block may also be integrally formed.

The solenoid coil 30 includes a first coil 31 and a second coil 32. The first coil 31 and the second coil 32 are spaced apart from each other by a certain distance in a longitudinal direction of the body 10.

The first coil 31 is a coil that is wound on the front of an outer circumferential surface of the body 10 and to which a current is applied when the piston 40 moves forward. The first coil 31 generates a magnetic force in a direction in which the moving magnetic body 90 moves forward, when a current is applied to the first coil 31, so as to move the moving magnetic body 90 forward.

The second coil 32 is a coil that is wound on the rear of an outer circumferential surface of the body 10, i.e., behind the first coil 31, and to which a current is applied when the piston 40 moves forward.

Current is applied to the second coil 32 to generate a magnetic force in an opposite direction to the direction of a magnetic field of the stationary magnet 92 when the piston 40 moves forward, so as to offset the magnetic field of the stationary magnet 92.

The supply of current to the second coil 32 is cut off when the piston 40 moves backward, so as to restore the magnetic field of the stationary magnet 92.

The piston 40 is long inserted into the body 10 and the cylinder 20 in the longitudinal direction and pushes the drug accommodated in the drug accommodating portion 22.

The piston 40 is provided separately from the moving magnetic body 90 inside the body 10 and is inserted in front of the moving magnetic body 90.

A flange portion 43 protruding in a radial direction is formed on the outer circumferential surface of the piston 40 to limit a moving distance of the piston 40.

The flange portion 43 is blocked by a length adjustment blocker 72 to be described later during the forward movement of the piston 40 so that a forward movement distance of the piston 40 is limited.

The blocker 70 is detachably coupled between the cylinder 20 and the piston 40.

The blocker 70 includes a fixed blocker 71 coupled to and fixed to the cylinder main hole 21, and a length adjustment blocker 72 that is screwed to an inner circumferential surface of the fixed blocker 71 and adjusts a length at which the length adjustment blocker 72 is coupled to the fixed blocker 71.

The fixed blocker 71 has a female thread formed on an inner circumferential surface of the fixed blocker 71 and formed in a ring shape.

The length adjustment blocker 72 has a male thread on an outer circumferential surface of the length adjustment blocker 72 and formed in a ring shape. A predetermined interval is formed between the length adjustment blocker 72 and the piston 40, and the piston 40 may pass through the inside of the length adjustment blocker 72 to move forward and backward.

The length adjustment blocker 72 is screwed at the rear of the fixed blocker 72, and a coupling length at which the length adjustment blocker 72 is screw-coupled to the fixed blocker 72, may be adjusted differently depending on one injection amount of the drug.

As the length of the length adjustment blocker 72 screwed to the fixed blocker 71 increases, the length at which the length adjustment blocker 72 protrudes backward decreases. When the length of the length adjustment blocker 72 protruding backward decreases, a distance d between the length adjustment blocker 72 and the first flange portion 41 increases, so that a forward movement distance d of the piston 40 increases. As the forward movement distance d of the piston 40 increases, one injection amount of the drug is increased.

As the coupling length at which the length adjustment blocker 72 is screwed to the fixed blocker 71 decreases, the length at which the length adjustment blocker 72 protrudes backward increases. As the length at which the length adjustment blocker 72 protruding backward increases, the distance d between the length adjustment blocker 72 and the flange portion 43 decreases, so that the forward movement distance d of the piston 40 decreases. As the forward movement distance d of the piston 40 decreases, one injection amount of the drug is reduced.

Thus, the user may finely adjust one injection amount of the drug by adjusting the length at which the length adjustment blocker 72 is coupled to the fixed blocker 71.

The moving magnetic body 90 is inserted into the body 10 in a longitudinal direction and reciprocates forward and backward by a magnetic force generated when a current is applied to the first coil 31.

The moving magnetic body 90 is not a permanent magnet, but is formed of a material that temporarily has magnetism by a magnetic field generated when a current is applied to the first coil 31 and disappears when the external magnetic field disappears. A case where the moving magnetic body 90 is an iron core, will be described.

The stationary magnet 92 is long inserted and fixed in a longitudinal direction behind the moving magnetic body 90 inside the body 10.

The stationary magnet 92 is a permanent magnet. The stationary magnet 92 is a material in which magnetism remains even when the external magnetic field disappears.

The stationary magnet 92 losses magnetism when a current is applied to the second coil 32 during the forward movement of the piston 40, a direction of a magnetic field generated around the second coil 32 is opposite to the direction of the magnetic field of the stationary magnet 92 and thus the magnetic field is offset, and the stationary magnet 92 does not serve as a magnet, and restores a magnet function again when the supply of a current to the second coil 32 is cut off during the backward movement of the piston 40. The stationary magnet 92 pulls the moving magnetic body 90 in a direction of backward movement due to electrical attraction so that the piston 40 can be moved backward.

The nozzle portion opening/closing valve 50 is provided to open and close a passage hole between the nozzle portion 23 and the drug accommodating portion 22. The nozzle portion opening/closing valve 50 is pushed by a fluidic pressure applied by the drug accommodated in the drug accommodating portion 22 during the forward movement of the piston 40 to open the passage hole, and is elastically restored when the fluidic pressure is released to close the passage hole.

The nozzle portion opening/closing valve 50 includes a ball 51 installed in the passage hole, and an elastic member 52 that is installed in the nozzle portion 23 to provide an elastic force in a direction of the ball 51 toward the drug accommodating portion 22. The ball 51 is formed to fit into the enlarged portion 22b. In the present embodiment, the nozzle portion opening/closing valve 50 has been described as an example of a ball valve. However, the present invention is not limited thereto, and various valves such as a duckbill valve, a plate check valve, an electric control valve, and the like may be used as the nozzle portion opening/closing valve 50.

The forward/backward driving unit may repeat the supply and cut-off of current to the first coil 31 and the second coil 32 at a preset period, thereby repeating the forward movement and the backward movement of the moving magnetic body 90 a plurality of times.

The forward/backward driving unit includes a current supply unit (not shown) and the elastic member 80.

The elastic member 80 is installed between the body 10 and the flange portion 43 of the piston 40.

The elastic member 80 is a spring that is compressed when the piston 40 moves forward and applies an elastic force to the piston 40 in the backward movement direction of the piston 40 when the supply of current to the first coil 31 is cut off.

The current supply unit (not shown) applies a current to each of the first coil 31 and the second coil 32 when the piston 40 moves forward, thereby moving the moving magnetic body 90 forward. When the piston 40 moves backward, the current supply unit (not shown) cuts off the current to each of the first coil 31 and the second coil 32, thereby moving the moving magnetic body 90 backward due to a magnetic field of the stationary magnet 92.

The current supply unit (not shown) include a capacitor (not shown) that is connected to the first coil 31 and stores the current supplied from an external power supply source, and a direct current (DC) power supply unit (not shown) that is connected to the second coil 32 and supplies the current supplied from the external power supply source to the second coil. However, the present invention is not limited thereto, and the capacitor (not shown) may be connected to both the first coil 31 and the second coil 32, or the capacitor (not shown) may also be connected to the second coil 32 and the DC power supply unit (not shown) may also be connected to the first coil 31.

The capacitor (not shown) supplies the stored current to the first coil 31 when the moving magnetic body 90 moves forward and discharges, and when the moving magnetic body 90 moves backward, the capacitor (not shown) stores the current and charges without supplying a current to the first coil 31. Thus, because an electrical energy applied during the forward movement of the moving magnetic body 90 is greater than the electrical energy applied during the backward movement, forward movement speed may be increased.

The operation of the needleless syringe 100 according to the first embodiment of the present invention configured as described above will be described as follows.

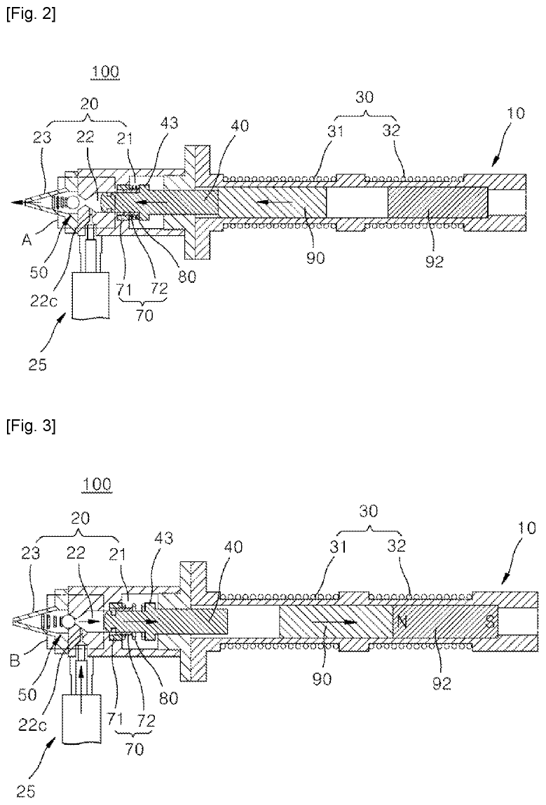

FIG. 2 is a view showing a forward movement state of a piston of the needleless syringe according to the first embodiment of the present invention. FIG. 3 is a view showing a backward movement state of the piston of the needleless syringe according to the first embodiment of the present invention.

First, an operation of moving the piston 40 forward will be described.

Referring to FIG. 2, the current supply unit (not shown) applies currents to both the first coil 31 and the second coil 32.

In this case, times at which a current is applied to each of the first coil 31 and the second coil 32, are differently set.

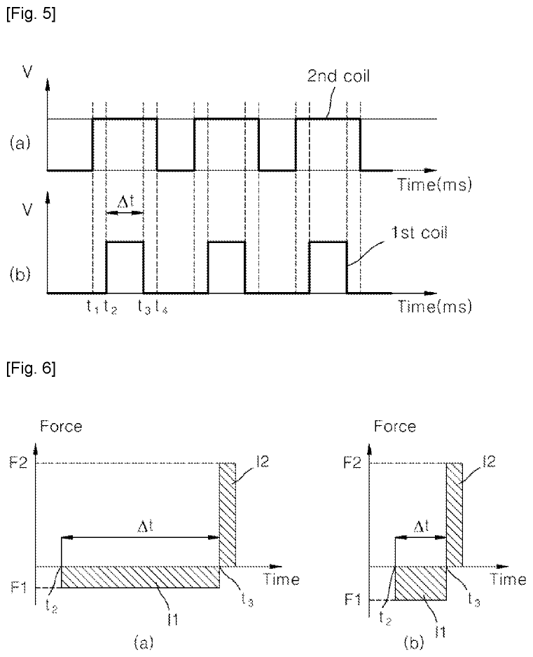

FIG. 5 shows a current supply waveform applied to each of the first coil 31 and the second coil 32.

Referring to FIG. 5, the current supply unit (not shown) first applies a current to the second coil 32 and then, when a second preset time (t2−t1) elapses, applies a current to the first coil 31.

In FIG. 5, t1 is a first time at which a current is applied to the second coil 32, and t2 is a second time at which a current is applied to the first coil 31, t3 is a third time at which the supply of current to the first coil 31 is cut off, and t4 is a fourth time at which the supply of current to the second coil 31 is cut off.

When a current is applied to the second coil 32 at the first time t1, a magnetic field in an opposite direction to the direction of the magnetic field of the stationary magnet 92 is generated around the second coil 32. That is, the direction of current is pre-set so that a magnetic field in the opposite direction to the direction of the magnetic field of the stationary magnet 92 can be generated in the second coil 32.

When the magnetic field in the opposite direction to the direction of the magnetic field of the stationary magnet 92 by the current applied to the second coil 32, the magnetic field of the stationary magnet 92 and the magnetic field of the second coil 32 have different directions from each other and are offset each other.

Thus, the stationary magnet 92 loses magnetism temporarily and thus electrical attraction between the stationary magnet 92 and the moving magnetic body 90 disappears. Since force of the stationary magnet 92 for pulling the moving magnetic body 90 disappears, the moving magnetic body 90 is in a forward movement state. The second preset time (t2−t1) corresponds to time for the moving magnetic body 90 prepares for forward movement.

When the second preset time (t2−t1) elapses from the first time t1, the current supply unit (not shown) applies a current to the first coil 31 at the second time t2. In this case, a state in which a current is supplied to the second coil 32, is also maintained.

By leaving a time difference between the time t1 at which the supply of current to the second coil 32 starts and the time t2 at which the supply of current to the first coil 31 starts, the magnetism of the stationary magnet 92 may be lost and the moving magnetic body 90 may be separated from the stationary magnet 92, and then the moving magnetic body 90 may be moved forward. Thus, compared to the case where the moving magnetic body 90 is moved forward by simultaneously applying current to the first coil 31 and the second coil 32, it is easier to move the moving magnetic body 90 forward while reducing the current applied to the first coil 31.

When a current is applied to the first coil 31, a magnetic force between the first coil 31 and the moving magnetic body 90 is generated.

By the magnetic force generated by the first coil 31, the moving magnetic body 90 moves forward.

When the moving magnetic body 90 moves forward by a certain distance or more, the moving magnetic body 90 collides with the piston 40.

The piston 40 is moved forward by an impact force applied when the moving magnetic body 90 collides with the piston 40.

The impact force is proportional to the mass and the moving speed of the moving magnetic body 90. The moving speed may be adjusted according to voltage applied to the first coil 31 and the moving distance of the moving magnetic body 90. When the impact force is changed by adjusting the voltage, one injection amount of the drug can be adjusted.

In the present embodiment, since the piston 40 moves forward due to the impact force, the piston 40 can be moved forward at higher speed compared to the case where the piston 40 and the moving magnetic body 90 are integrally moved forward. Thus, an electrical energy required to move the piston 40 forward can be further reduced.

When the piston 40 moves forward, the elastic member 80 is compressed.

The piston 40 may only move forward until the flange portion 43 is blocked by the length adjustment block 72.

When the piston 40 moves forward, the piston 40 pressurizes the drug in the drug accommodating portion 22.

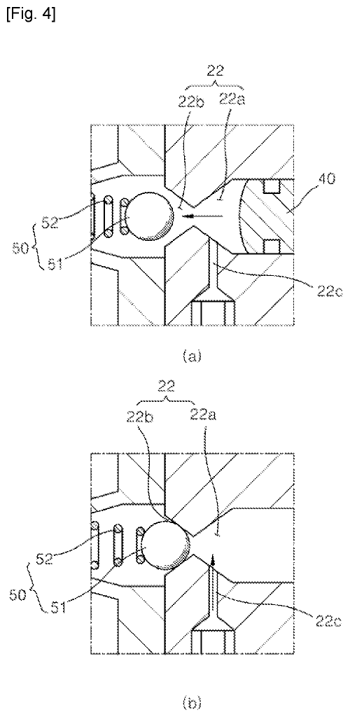

Referring to FIG. 4A, when the drug in the drug accommodating portion 22 is pressurized, the nozzle portion opening/closing valve 50 is opened by the fluidic pressure.

When the nozzle portion opening/closing valve 50 is opened, the drug in the drug accommodating portion 22 may be injected forward through the nozzle portion 23.

When the piston 40 moves forward, as described above, because the piston 40 may only move forward until the flange portion 43 is blocked by the length adjustment blocker 72, the forward movement distance of the piston 40 is limited.

Thus, a coupling length at which the length adjustment blocker 72 is coupled to the fixed blocker 71, is adjusted to adjust the length at which the length adjustment blocker 72 protruding backward toward the flange portion 43 so that the forward movement distance of the piston 40 can be adjusted. By adjusting the forward movement distance of the piston 40, one injection amount of the drug may be adjusted.

That is, one injection amount of the drug is adjustable according to the magnitude of the voltage applied to the first coil 31 and the forward movement distance of the piston 40.

Subsequently, the current supply unit supplies a current to the first coil 31 for a first preset time Δt, and then, when the first preset time Δt elapses, the supply of current to the first coil 31 is cut off.

Referring to FIG. 5, the current supply unit (not shown) first cuts off current to the first coil 31 and then, when a third preset time (t4−t3) elapses, the current supply unit (not shown) cuts off current to the second coil 32.

When the first preset time Δt elapses and it becomes the third time t3, the supply of current to the first coil 31 is cut off.

The first preset time Δt is set to be about 180 ms or less. A description of the first preset time Δt will be provided in detail.

When the supply of current to the first coil 31 is cut off, a magnetic field by the first coil 31 disappears, and a force for moving the moving magnetic body 90 forward disappears.

When the third preset time (t4−t3) elapses and it becomes the fourth time t4, the supply of current to the second coil 32 is cut off.

When the supply of current to the second coil 32 is cut off, the magnetic field around the second coil 32 disappears, and the magnetic field of the stationary magnet 92 is restored, and the stationary magnet 92 generates a magnetic force in a direction in which the moving magnetic body 90 moves backward.

Thus, the moving magnetic body 90 moves backward by a force pulled by the stationary magnet 92 and is attached to the stationary magnet 92.

When the moving magnetic body 90 moves backward, a force for the moving magnetic body 90 applied to the piston 40 disappears.

Thus, the piston 40 may move backward to its original position by an elastic restoration force of the elastic member 80.

Referring to FIG. 4B, when the piston 40 moves backward, pressure in the drug accommodating portion 22 is lowered, and the nozzle portion opening/closing valve 50 is elastically restored and is closed.

In addition, when the pressure in the drug accommodating portion 22 is lowered, the drug may be filled in the drug accommodating portion 22 from the drug filling device 25 through the drug supply hole 22c. That is, when the piston 40 moves backward, the drug may be automatically filled.

Meanwhile, the current supply unit may supply a current to the first coil 31 for a first preset time Δt and then cuts off the current when the first preset time Δt elapses.

The first preset time Δt is a time difference from the time t2 at which a current is applied to the first coil 31, to the time t3 at which the supply of current to the first coil 31 is cut off.

The first preset time Δt is set to time at which a first recoil impulse 11 generated during the forward movement of the moving magnetic body 90 and a second recoil impulse 12 generated when the moving magnetic body 90 collides with the piston 40 may be offset each other.

When the moving magnetic body 90 moves forward, first recoil is generated in the needleless syringe 100 or the user's hand holding the needleless syringe 100 in the backward movement direction that is opposite to the forward movement direction according to the law of action and reaction.

When the moving magnetic body 90 collides with the piston 40, second recoil is generated in the needleless syringe 100 or the user's hand holding the needleless syringe 100 in the forward movement direction according to the law of action and reaction.

Referring to FIG. 6, 11 represents a first recoil impulse generated during the first recoil, and 12 represents a second recoil impulse generated during the second recoil.

The first recoil impulse 11 and the second recoil impulse 12 generated by a difference of the first preset time Δt are the same.

Thus, when the first preset time Δt is reduced, the user feels that the first recoil impulse 11 and the second recoil impulse 12 are almost simultaneously generated, and thus, the effect of offsetting the first recoil impulse 11 and the second recoil impulse 12 can be achieved. That is, as shown in FIG. 6B, the first preset time Δt is reduced and is derived as an optimum time at which recoil can be offset, so that a recoil offset effect can be attained.

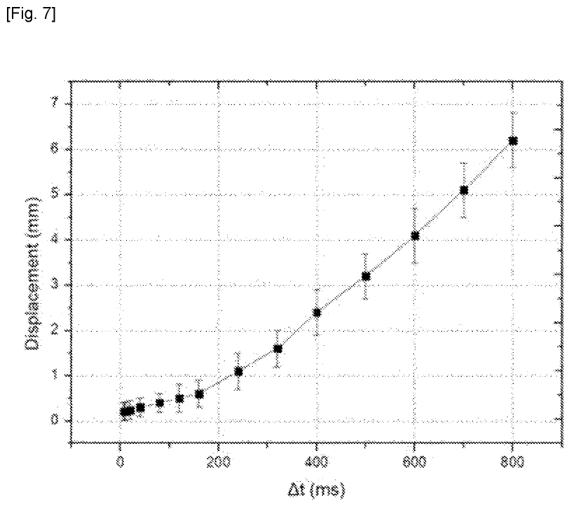

FIG. 7 is a graph showing a recoil distance (Displacement) according to time at which a current is applied to the first coil of the needless syringe according to the first embodiment of the present invention.

Referring to FIG. 7, experiments in which a recoil distance is measured by changing the first preset time so as to derive the optimum value of the first preset time Δt, have been carried out.

In the experiments, the first preset time Δt was gradually reduced from 800 ms, and the moving distance of the moving magnetic body was fixed to 60 mm. The weight of the moving magnetic body was reduced according to a reduction in the first preset time Δt so that the impulse of the moving magnetic body 90 was maintained.

Here, the recoil distance is a moving distance of the needleless syringe during one injection of the drug. As the recoil distance decreases, a recoil impact felt by the user is reduced.

When voltage applied to the first coil 31 increases, the moving speed of the moving magnetic body 90 increases and thus, the first preset time Δt can be reduced.

In this case, when the moving speed of the moving magnetic body 90 increases, the impulse applied to the piston 40 increases and thus, the mass of the moving magnetic body 90 compared to the moving speed needs to be reduced so as to maintain the impulse at a constant level. Since the mass of the moving magnetic body 90 can be adjusted in length or reduced in cross-sectional area, it is preferable to adjust the mass by forming a hole inside rather than adjusting the length.

In the present experiments, the voltage applied to the first coil 31 increased to reduce the first preset time, wherein the mass of the moving magnetic body 90 was reduced to maintain the impulse applied to the piston 40.

As the result of the experiments, referring to FIG. 7, there is little change in recoil distance when the first preset time is 180 ms or less.

Thus, the first preset time is 180 ms or less, and the optimum weight of the moving magnetic body 90 caused by a reduction in the first preset time is 30 g.

Time at which a current is applied to the first coil 31, is set to be 180 ms or less so that recoil felt by the user when using the needleless syringe 100 is offset, and convenience of use can be enhanced.

As described above, in the present invention, current is selectively supplied or cut off to the first coil 31 and the second coil 32 so that the forward movement and the backward movement of the piston 40 can be repeated.

In addition, because a small amount of the drug can be repeatedly injected at high speed, a small amount of the drug can be injected into the entire skin several times in the field of skin care and the like.

In addition, in the present invention, since the piston 40 moves forward due to the impulse, the piston 40 can move forward at a higher speed compared to the case where the piston 40 and the moving magnetic body 90 move forward integrally. Thus, an electrical energy required to move the piston 40 forward can be further reduced.

In addition, time at which a current is applied to the first coil 31, is adjusted so that the recoil of the needleless syringe 100 can be minimized, and thus, since there is no need to add an additional recoil offset structure, the structure can be simplified, and convenience of use can be enhanced.

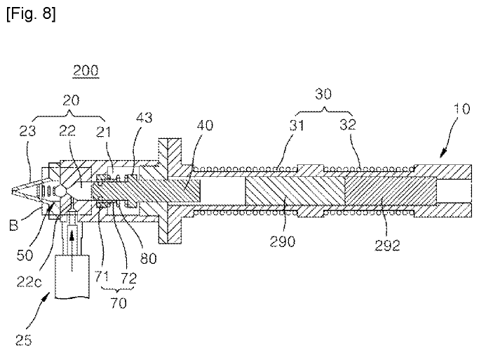

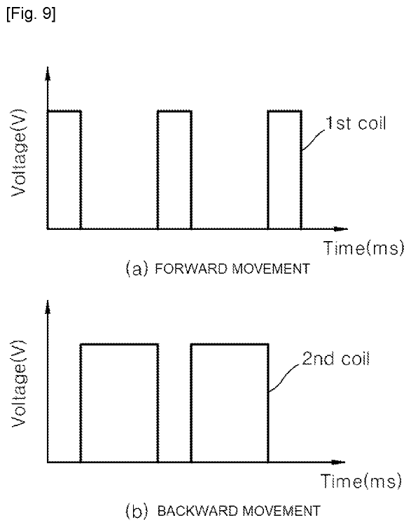

FIG. 8 is a longitudinal perspective view showing a needleless syringe according to a second embodiment of the present invention. FIG. 9 is a graph showing a current supply waveform applied to each of a first coil and a second coil of the needleless syringe according to the second embodiment of the present invention.

Referring to FIG. 8, a needleless syringe 200 according to the second embodiment of the present invention is different from that of the first embodiment in that the needleless syringe 200 according to the second embodiment of the present invention includes a moving magnetic body 290 and a stationary magnetic body 292 provided inside the body 10, the moving magnetic body 290 and the stationary magnetic body 292 have magnetism only when currents are applied to the first coil 31 and the second coil 32 and thus, a current is applied only to the first coil 31 and the supply of current to the second coil 32 is cut off when the piston 40 moves forward, and the rest of the configuration and operation thereof is similar to those of the first embodiment, and thus, a description of the similar configurations will be omitted, and different configurations will be mainly described below.

The moving magnetic body 290 is long inserted into the body 10 in the longitudinal direction, and reciprocates forward and backward due to a magnetic force generated when a current is applied to the first coil 31. The moving magnetic body 290 is not a permanent magnet, but is formed of a material which temporarily has magnetism by a magnetic field generated when a current is applied to the first coil 31 and in which magnetism disappears when the external magnetic field disappears. A case where the moving magnetic body 290 is an iron core, will be described.

The stationary magnetic body 292 is inserted and fixed into the body 10 in a position where the stationary magnetic body 292 is spaced apart from the moving magnetic body 90 backward.

The stationary magnetic body 292 is magnetized by the magnetic force generated when a current is applied to the second coil 32 and has a polarity. When the stationary magnetic body 292 has a polarity, the moving magnetic body 290 is pulled in a direction of backward movement by electrical attraction. The stationary magnetic body 292 is not a permanent magnet and is formed of a material which temporarily has magnetism by the magnetic field generated when a current is applied to the second coil 32 and in which magnetism disappears when the external magnetic field disappears. A case where the stationary magnetic body 292 is an iron core, will be described.

The current supply unit (not shown) supplies a current only to the first coil 31 and cuts off the supply of current to the second coil 32 when the piston 40 moves forward, and the current supply unit (not shown) cuts off the supply of current to the first coil 31 and supplies a current to the second coil 32 when the piston 40 moves backward.

The current supply unit (not shown) includes a capacitor (not shown) that is connected to the first coil 31 and stores current supplied from an external power supply source, and a DC power supply unit (not shown) that is connected to the second coil 32 and supplies the current supplied from the external power supply source to the second coil.

The capacitor (not shown) supplies the stored current to the first coil 31 when the moving magnetic body 290 moves forward and discharges, and when the moving magnetic body 290 moves backward, the capacitor (not shown) stores the current and charges without supplying a current to the first coil 31. Thus, because an electrical energy applied during the forward movement of the moving magnetic body 290 is greater than the electrical energy applied during the backward movement, forward movement speed may be increased.

The operation of the needleless syringe according to the second embodiment of the present invention having the above configuration will be described as follows.

When the piston moves forward, the current supply unit applies a current to the first coil 31 and cuts off the supply of current to the second coil 32.

When a current is supplied to the first coil 31, a magnetic force between the first coil 31 and the moving magnetic body 290 is generated.

By the magnetic force generated by the first coil 31, the moving magnetic body 290 moves forward.

When the moving magnetic body 290 moves forward by a certain distance or more, the moving magnetic body 290 collides with the piston 40.

The piston 40 moves forward by an impulse applied when the moving magnetic body 290 collides with the piston 40.

The impulse may be differently adjusted according to the mass and moving speed of the moving magnetic body 290. The magnetic force may be adjusted according to a voltage applied to the first coil 31. By changing the magnetic force, one injection amount of the drug can be adjusted.

In the present invention, since the piston 40 moves forward by the impulse, the piston 40 may move forward at a higher speed compared to the case where the piston 40 and the moving magnetic body 290 move forward integrally. Thus, an electrical energy required to move the piston 40 forward can be further reduced.

When the piston 40 moves forward, the elastic member 80 is compressed.

The piston 40 may move forward only until the flange portion 43 is blocked by the length adjustment blocker 72.

When the piston 40 moves forward, the piston 40 pressurizes the drug in the drug accommodating portion 22.

When the drug in the drug accommodating portion 22 is pressurized, the nozzle portion opening/closing valve 50 is opened by a fluidic pressure.

When the nozzle portion opening/closing valve 50 is opened, the drug in the drug accommodating portion 22 may be injected forward through the nozzle portion 23.

When the piston 40 moves forward as described above, the piston 40 may move forward only until the flange portion 43 is blocked by the length adjustment blocker 72 so that the forward movement distance of the piston is limited.

Thus, the length adjustment blocker 72 adjusts a length protruding backward toward the flange portion 43 by adjusting a coupling length at which the length adjustment blocker 72 is coupled to the fixed blocker 71, so that the forward movement distance of the piston can be adjusted. When the forward movement distance of the piston 40 is adjusted, the amount of the drug injected one time can be adjusted.

That is, one injection amount of the drug is adjustable according to the magnitude of a voltage applied to the first coil 31 and the forward movement distance of the piston 40.

Meanwhile, the current supply unit supplies a current to the first coil 31 for a first preset time Δt and then cuts off the supply of current when the first preset time Δt elapses.

When the first preset time Δt elapses, the forward/backward driving unit cuts off the supply of current to the first coil 31 and supplies a current to the second coil 32 to move the piston 40 backward.

The first preset time Δt is set to be about 80 ms or less.

The first preset time Δt is set to time at which the first recoil impulse 11 generated when the moving magnetic body 90 moves forward and the second recoil impulse 12 generated when the moving magnetic body 290 collides with the piston 40, are offset each other.

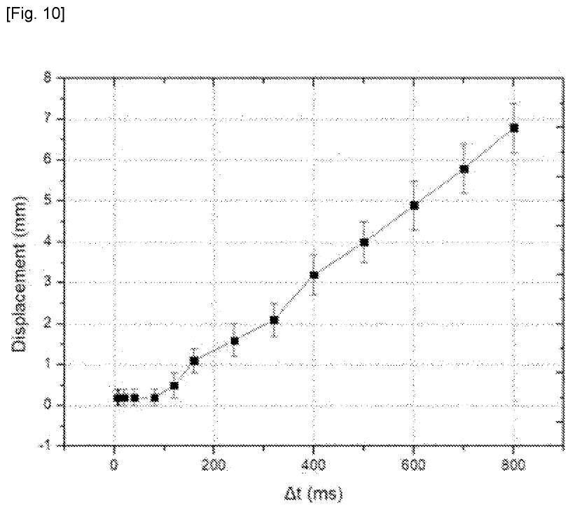

FIG. 10 is a graph showing a recoil distance (Displacement) according to time at which a current is applied to the first coil of the needless syringe according to the second embodiment of the present invention.

Referring to FIG. 10, experiments for measuring a recoil distance by changing the first preset time so as to derive the optimum value of the first preset time Δt were carried out on the needleless syringe 200.

Since experimental conditions are the same as those of the first embodiment, detailed descriptions thereof will be omitted.

As the result of the experiments, referring to FIG. 10, since there is little change in recoil distance when the first preset time Δt is 80 ms or less, the first preset time Δt is set to 80 ms. In this case, the optimum weight of the moving magnetic body 290 according to a reduction of the first preset time Δt is 100 g.

Thus, time at which a current is applied to the first coil 31 is set to 80 ms or less, and the weight of the moving magnetic body 290 is set to 100 g so that recoil felt by the user when the needleless syringe 100 is used, may be offset and convenience of use may be enhanced.

When the first preset time Δt elapses, the supply of current to the first coil 31 is cut off, and a current is supplied to the second coil 32.

When a current is supplied to the second coil 32, a magnetic force between the second coil 32 and the stationary magnetic body 292 is generated.

The stationary magnetic body 292 has a polarity due to the magnetic force and is magnetized.

In addition, when the supply of current to the first coil 31 is cut off, the magnetic force by the first coil 31 disappears, and thus, the moving magnetic body 290 loses a polarity temporarily but has a polarity again due to the magnetic force generated by the second coil 32.

In this case, the moving magnetic body 290 is affected by the magnetic force caused by the second coil 31 and thus has the same polarity as that of the stationary magnetic body 292.

Electrical attraction is generated between the stationary magnetic body 292 and the moving magnetic body 290 so that the stationary magnetic body 292 pulls the moving magnetic body 290. The stationary magnetic body 292 is fixed installed and thus does not move.

Thus, the moving magnetic body 290 moves backward by force pulled by the stationary magnetic body 292.

When the moving magnetic body 290 moves backward, force applied to the piston 40 by the moving magnetic body 290 disappears.

Thus, the piston 40 may move backward to its original position by the elastic restoration force of the elastic member 80.

When the piston 40 moves backward, the pressure in the drug accommodating portion 22 is lowered and thus the nozzle portion opening/closing valve 50 is elastically restored and is closed.

In addition, when the pressure in the drug accommodating portion 22 is lowered, the drug may be filled in the drug accommodating portion 22 through the drug supply hole 22c from the drug filling device 25. That is, when the piston 40 moves backward, the drug may be automatically filled.

Meanwhile, the current supply unit supplies a current to the second coil 32 for a second preset time and then cuts off the current when the second preset time elapses.

Since a current is applied to the second coil 32 so as to move the piston 40 backward, a magnetic force generated by the second coil 32 is set to be smaller than a magnetic force generated by the first coil 31. Thus, since a weaker magnetic force is generated around the second coil 32, time at which a current is applied to the second coil 32 so as to move the piston 40 backward is set to be longer than time at which a current is applied to the first coil 31. In addition, time at which a current is applied to the second coil 32, may be increased/decreased according to a number of repeated injections per second of the drug. For example, when the number of repeated injections per second of the drug is increased to 50 to 100 Hz or more, time at which a current is applied to the second coil 32 may be set to be shorter.

When the second preset time elapses, the current supply unit cuts off the supply of current to the second coil 32 and supplies a current to the first coil 31, thereby moving the moving magnetic body 290 forward again.

As described above, a current is selectively supplied or cut off to one of the first coil 31 and the second coil 32 so that the forward movement and the backward movement of the piston 40 can be repeated.

Thus, since a small amount of the drug can be repeatedly injected at a high speed, a small amount of the drug can be injected onto the entire skin several times in the fields of skin care or the like.

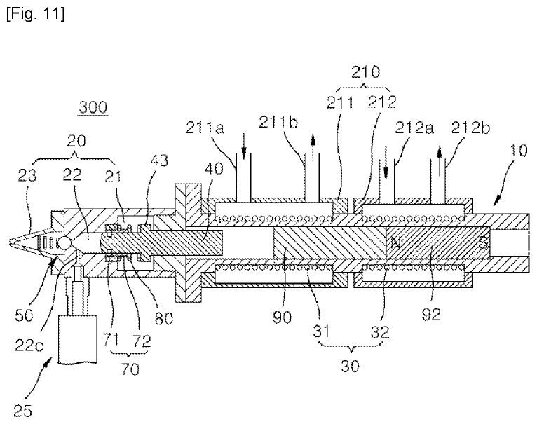

FIG. 11 is a longitudinal cross-sectional view showing a configuration in which a cooling chamber is provided in a needless syringe according to a third embodiment of the present invention.

Referring to FIG. 11, a needleless syringe 300 according to the third embodiment of the present invention is different from the first and second embodiments in that the needleless syringe 300 according to the third embodiment of the present invention further includes a cooling chamber 210 for cooling heat generated in the solenoid coil 30 through a cooling fluid, and the rest of the configuration and operation thereof is similar to those of the first and second embodiments, and thus, a description of the similar configurations will be omitted, and different configurations will be mainly described below.

The cooling chamber 210 is detachably coupled to the outside of the body 10.

The cooling chamber 210 includes a first cooling chamber 211 installed to surround the first coil 31 on the outer circumferential surface of the body 10, and a second cooling chamber 212 installed to surround the second coil 32 on the outer circumferential surface of the body 10.

A first cooling fluid supply pipe 211a and a first cooling fluid discharge pipe 211b are coupled to the first cooling chamber 211.

The first cooling fluid supply pipe 211a is a flow path for supplying a cooling fluid from the outside to the first cooling chamber 211.

The first cooling fluid discharge pipe 211b is a flow path for discharging the cooling fluid of the first cooling chamber 211 to the outside.

An opening/closing valve (not shown) may be provided in the first cooling fluid supply pipe 211a and the first cooling fluid discharge pipe 211b, respectively.

A second cooling fluid supply pipe 212a and a second cooling fluid discharge pipe 212b are coupled to the second cooling chamber 212.

The second cooling fluid supply pipe 212a is a flow path for supplying the cooling fluid to the second cooling chamber 212 from the outside.

The second cooling fluid discharge pipe 212b is a flow path for discharging the cooling fluid of the second cooling chamber 212 to the outside.

An opening/closing valve (not shown) may be provided in the second cooling fluid supply pipe 212a and the second cooling fluid discharge pipe 212b, respectively.

In the present embodiment, a case where two cooling chambers 210 are provided, has been described, but the present invention is not limited thereto, and of course, one cooling chamber 210 may also be provided to surround both the first coil 31 and the second coil 32.

In addition, in the present embodiment, a cooling fluid is used to cool the solenoid coil 30, and water or air is used as the cooling fluid. However, the present invention is not limited, and a conduction cooling method or the like may be used.

The needleless syringe 300 according to the third embodiment of the present invention configured as described above is provided with the cooling chamber 210 for cooling the solenoid coil 30, so that heat of the solenoid coil 30 can be absorbed and the solenoid coil can be kept at a constant temperature and thus, the magnetic force can be prevented from being weakened by the heat generated by the solenoid coil 30.

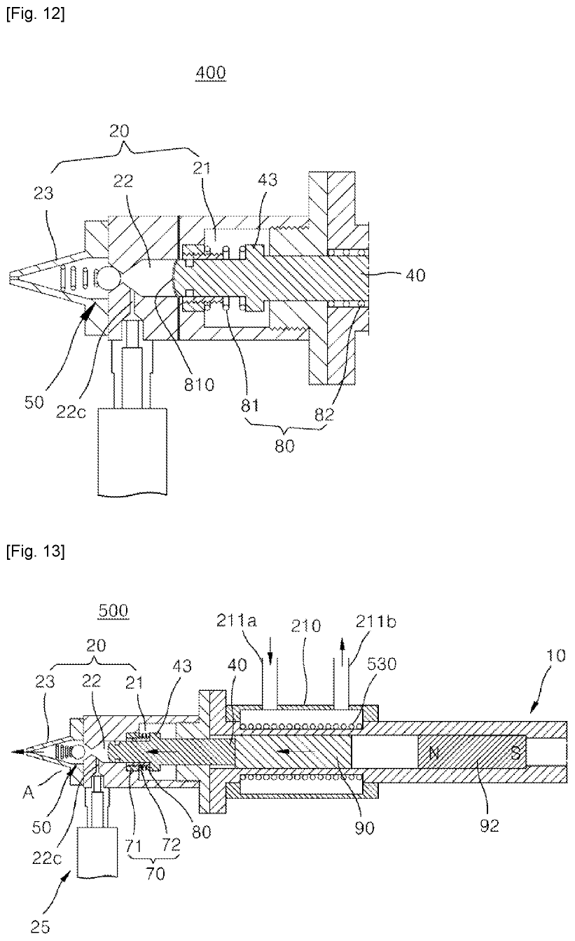

FIG. 12 is a longitudinal cross-sectional view showing a configuration in which a piston cover is provided in a needless syringe according to a fourth embodiment of the present invention.

Referring to FIG. 12, a needleless syringe 400 according to the fourth embodiment of the present invention is different from the above-described embodiments in that a piston cover 810 is provided between the cylinder 20 and the piston 40, and the rest of the configuration and operation thereof is similar to those of the above-described embodiments, and thus, a description of the similar configurations will be omitted, and different configurations will be mainly described below.

The piston cover 810 is applicable to all of the first, second and third embodiments.

The piston cover 810 is fixedly installed at the cylinder 20. The piston cover 810 is provided inside the cylinder 20 and is disposed to cover the end portion of the piston 40. The piston cover 810 may be inserted into the cylinder 20 and coupled thereto, or may also be fixed to the inner circumferential surface of the cylinder 20 through a method such as adhesion or coupling or the like.

The piston cover 810 is formed of an elastic material to be elongated forward by the piston 40 when the piston 40 moves forward and to be restored when the piston 40 moves backward. In the present embodiment, a case where the piston cover 810 is a rubber film, will be described.

The piston cover 810 may prevent the drug from directly staining the end portion of the piston 40.

Thus, there is no need for the user to wipe the end portion of the piston 40.

In addition, the piston cover 810 is provided at the cylinder 20 so that the piston cover 810 may also be replaced when the cylinder 20 is replaced.

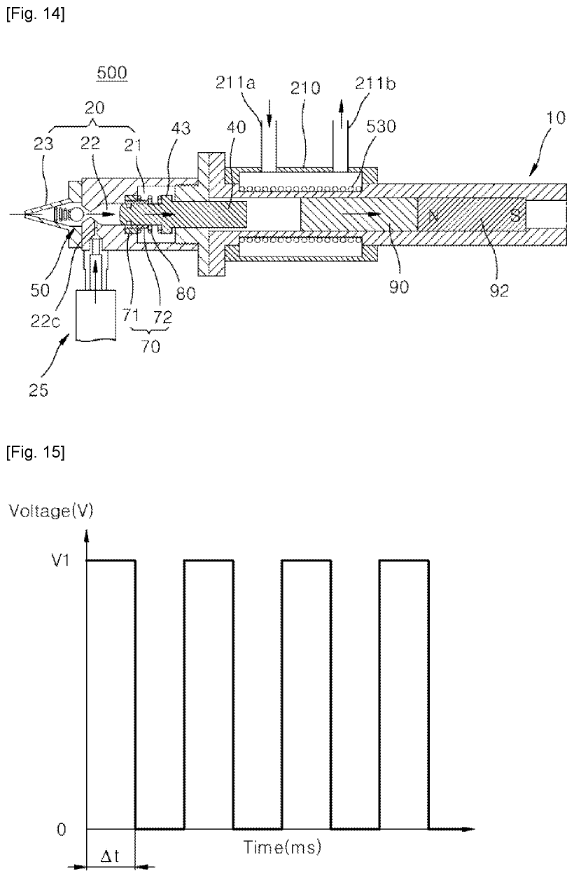

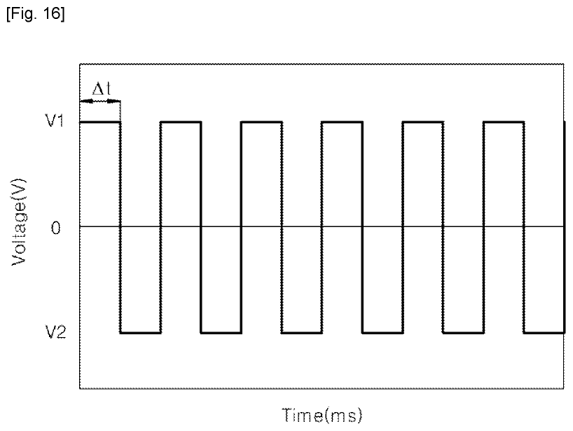

FIG. 13 is a view showing a forward movement state of a piston of the needleless syringe according to a fifth embodiment of the present invention. FIG. 14 is a view showing a backward movement state of the piston of the needleless syringe according to the fifth embodiment of the present invention. FIG. 15 is a graph showing another example of a current supply waveform applied to a solenoid coil of a needleless syringe according to the fifth embodiment of the present invention.

Referring to FIGS. 13 and 14, a needleless syringe 500 according to the fifth embodiment of the present invention is different from the above-described embodiments in that only one solenoid coil 530 is provided, a forward/backward moving unit for moving the piston 40 forward/backward includes a current supply unit (not shown) for generating a greater magnetic force than a magnetic force of the stationary magnet 92 by supplying a current to the solenoid coil 530 when the piston 40 moves forward, and the rest of the configuration and operation thereof is similar to those of the above-described embodiments, and thus, a description of the similar configurations will be omitted, and different configurations will be mainly described below.

The current supply unit supplies a current to the solenoid coil 530 when the piston 40 moves forward.

The current supply unit supplies a current to the solenoid coil 530 so that a first magnetic force generated around the solenoid coil 530 and the moving magnetic body 90 is greater than a second magnetic force of the stationary magnet 92. When a current applied to the solenoid coil 530 is increased, the first magnetic force may be greater than the second magnetic force. The current applied to the solenoid coil 530 may be preset by experiments or the like. In addition, a magnet having low magnetism may be used so that the second magnetic force of the stationary magnet 92 is smaller than the first magnetic force.

The needleless syringe 500 further includes a cooling chamber 210 for cooling heat generated in the solenoid coil 530 through a cooling fluid. The cooling chamber 210 is similar to that of the third embodiment and thus, a detailed description thereof will be omitted.

In addition, the needleless syringe 500 may further include a piston cover 810 described in the fourth embodiment.

The operation of the needleless syringe 500 according to the fifth embodiment of the present invention having the above configuration will be described as follows.

Referring to FIG. 13, when the piston 40 moves forward, a current having a preset first direction and a first magnitude are applied to the solenoid coil 530. Here, the first direction is set to a direction in which a magnetic force is generated in a direction of forward movement of the moving magnetic body 90 around the solenoid coil 530. In addition, the first magnitude is set so that the first magnetic force generated around the solenoid coil 530 is greater than the second magnetic force of the stationary magnet 92.

The second magnetic force of the stationary magnet 92 is a force of the stationary magnet 92 for pulling the moving magnetic body 90.

Since the first magnetic force generated by the solenoid coil 530 is greater than a force of the stationary magnet 92 for pulling the moving magnetic body 90, the moving magnetic body 90 may be separated from the stationary magnet 92 and may move forward.

When the moving magnetic body 90 moves forward by a certain distance or more, the moving magnetic body 90 collides with the piston 40.

The piston 40 moves forward by an impulse applied when the moving magnetic body 90 collides with the piston 40.

Referring to FIG. 14, when the piston 40 moves backward, the supply of current is to the solenoid coil 530 is cut off.

When the supply of current to the solenoid coil 530 is cut off, the first magnetic force around the solenoid coil 530 and the moving magnetic body 90 disappears, and only the second magnetic force of the stationary magnet 92 remains.

The second magnetic force of the stationary magnet 92 pulls the moving magnetic body 90 in a direction of backward movement.

Thus, the moving magnetic body 90 may move backward by the second magnetic force of the stationary magnet 90.

Meanwhile, FIG. 15 is a graph showing an example of a current supply waveform applied to a solenoid coil of the needleless syringe according to the fifth embodiment of the present invention.

Referring to FIG. 15, the current supply unit (not shown) applies a voltage of V1 to the solenoid coil 530 for the first preset time Δt when the piston 40 moves forward, and cuts off the voltage to the solenoid coil 530 when the piston 40 moves backward.

A case where time at which the voltage to the solenoid coil 530 is cut off, is set to be the same as time at which a voltage is applied to the solenoid coil 530, will be described.

FIG. 16 is a graph showing another example of a current supply waveform applied to a solenoid coil of a needleless syringe according to a sixth embodiment of the present invention.

Referring to FIG. 16, the needleless syringe according to the sixth embodiment of the present invention is different from the fifth embodiment in that a current supply unit changes the direction of a current applied to the solenoid coil 530 repeatedly at a preset period to change the direction of a magnetic force generated in the solenoid coil 530 periodically, and the rest of the configuration and operation thereof is similar to those of the above-described embodiments, and thus, a description of the similar configurations will be omitted, and different configurations will be mainly described below.

The current supply unit (not shown) supplies a current in a preset first direction to the solenoid coil 530 when the moving magnetic body 90 moves forward, and generates the first magnetic force that is greater than a second magnetic force of the stationary magnet 92.

The current supply unit (not shown) supplies a current in an opposite direction to the first direction to the solenoid coil 530 when the moving magnetic body 90 moves backward, and generates a magnetic force in a direction in which the moving magnetic body 90 moves backward.

Referring to FIG. 16, a case where, when the moving magnetic body 90 moves forward, a voltage of V1 that is a positive value is applied to the solenoid coil 530 for the first preset time Δt and when the moving magnetic body 90 moves backward, a voltage of V2 that is a negative value is applied to the solenoid coil 530 for the first preset time Δt, will be described. Since a larger magnetic force is required when the moving magnetic body 90 moves forward, an absolute value of V1 may be set to be larger than an absolute value of V2.

Thus, when the voltage of V1 is applied to the solenoid coil 530, a magnetic force is generated in a direction in which the moving magnetic body 90 moves forward, so that the moving magnetic body 90 may move forward.

In addition, when the voltage of V2 is applied to the solenoid coil 530, a magnetic force is generated in a direction in which the moving magnetic body 90 moves backward, so that the backward movement of the moving magnetic body 90 may be easily performed.

In addition, the present invention is not limited thereto, and the current supply unit (not shown) may include a capacitor (not shown) that stores current supplied from an external power supply source, supplies the stored current when the piston 40 moves forward and cuts off the supply of current to the solenoid coil 530 when the piston 40 moves backward, and a DC power supply unit that supplies current supplied from the external power supply source to the solenoid coil 530 when the piston 40 moves backward.

Thus, the current supply unit (not shown) may change the direction of current applied to the solenoid coil 30 at a preset period repeatedly, supply current stored in the capacitor (not shown) when the piston 40 moves forward, supply current from the DC power supply unit when the piston 40 moves backward, thereby increasing a moving speed when the piston 40 moves forward.

While the present invention has been particularly shown and described with reference to exemplary embodiments thereof, it will be understood by those of ordinary skill in the art that various changes in form and details may be made therein without departing from the spirit and scope of the present invention as defined by the following claims.

### INDUSTRIAL APPLICABILITY

According to the present invention, a needleless syringe capable of repeatedly injecting a certain drug at high speed can be manufactured.

1. A needleless syringe comprising:
a body;
a solenoid coil wound on an outer circumferential surface of the body;
a cylinder coupled to the body to be in communication with an open front surface of the body and comprising a drug accommodating portion in which the drug is accommodated, and a nozzle portion injecting the drug accommodated in the drug accommodating portion forward;
a moving magnetic body that is long inserted into the body in a longitudinal direction and reciprocates forward and backward by a magnetic force generated when a current is applied to the solenoid coil;
a stationary magnet that is inserted and fixed behind the moving magnetic body inside the body;
a piston that is inserted in front of the moving magnetic body inside the body and pressurizes the drug in the drug accommodating portion by an impulse applied by forward movement of the moving magnetic body when the moving magnetic body moves forward;
a nozzle portion opening/closing valve that is provided to open/close a passage hole between the nozzle portion and the drug accommodating portion; and
a forward/backward driving unit that repeats the supply and cut-off of current to the solenoid coil at a preset period, thereby repeating the forward movement and the backward movement of the piston,
wherein the solenoid coil comprises:
a first coil, which is wound on front of the outer circumferential surface of the body, to which a current is applied during forward movement of the piston and generates a magnetic force in a forward movement direction of the piston; and
a second coil, which is wound on rear of the first coil on the outer circumferential surface of the body, to which a current is applied during forward movement of the piston and which generates a magnetic force in an opposite direction to a direction of a magnetic field of the stationary magnet to offset the magnetic field of the stationary magnet and in which supply of current is cut off when the piston moves backward, to restore the magnetic field of the stationary magnet, and
the forward/backward driving unit comprises:
a current supply unit that repeatedly supplies a current to each of the first coil and the second coil during forward movement of the piston to move the moving magnetic body forward and repeatedly cuts off supply of current to each of the first coil and the second coil during backward movement of the piston to move the moving magnetic body backward due to the magnetic field of the stationary magnet.

2. The needleless syringe of claim 1, wherein the current supply unit applies a current to the first coil for a first preset time and then cuts off supply of current, and
a mass of the moving magnetic body is 100 g or less, and the first preset time is set to be 180 ms or less.

3. The needleless syringe of claim 1, wherein the current supply unit applies a current to the second coil and then, when a second preset time elapses, the current supply unit applies a current to the first coil, and when the piston moves backward, the current supply unit cuts off supply of current to the first coil and then, when a third preset time elapses, the current supply unit cuts off supply of current to the second coil.

4. A needleless syringe comprising:
a body;
a solenoid coil wound on an outer circumferential surface of the body;
a cylinder coupled to the body to be in communication with an open front surface of the body and comprising a drug accommodating portion in which the drug is accommodated, and a nozzle portion injecting the drug accommodated in the drug accommodating portion forward;
a moving magnetic body that is long inserted into the body in a longitudinal direction and reciprocates forward and backward by a magnetic force generated when a current is applied to the solenoid coil;
a stationary magnetic body that is inserted behind the moving magnetic body inside the body and has magnetism by a magnetic force generated when a current is applied to the solenoid coil and is fixed;
a piston that is inserted in front of the moving magnetic body inside the body and pressurizes the drug in the drug accommodating portion by an impulse applied by forward movement of the moving magnetic body when the moving magnetic body moves forward;
a nozzle portion opening/closing valve that is provided to open/close a passage hole between the nozzle portion and the drug accommodating portion; and
a forward/backward driving unit that repeats the supply and cut-off of current to the solenoid coil at a preset period, thereby repeating the forward movement and the backward movement of the piston,
wherein the solenoid coil comprises:
a first coil, which is wound on front of the outer circumferential surface of the body, to which a current is applied during forward movement of the piston and generates a magnetic force in a forward movement direction of the moving magnetic body; and
a second coil, which is wound on rear of the first coil on the outer circumferential surface of the body, to which a current is applied during backward movement of the piston and which generates a magnetic force to magnetize the stationary magnetic body, and
the forward/backward driving unit comprises:
a current supply unit that repeatedly supplies a current to the first coil to move the moving magnetic body forward and cuts off supply of current to the second coil during forward movement of the piston and that repeatedly cuts off supply of current to the first coil and supplies a current to the second coil to move the moving magnetic body backward due to the magnetic field of the stationary magnetic body during backward movement of the piston; and
the current supply unit applies a current to the first coil for a first preset time and then cuts off supply of current, and a mass of the moving magnetic body is 100 g or less, and the first preset time is set to be 80 ms or less.

5. The needleless syringe of claim 1, further comprising a cooling chamber that is provided to surround an outside of the solenoid coil on an outside of the body and absorbs and cools heat generated in the solenoid coil through a cooling fluid.

6. The needleless syringe of claim 4, further comprising a cooling chamber that is provided to surround an outside of the solenoid coil on an outside of the body and absorbs and cools heat generated in the solenoid coil through a cooling fluid.

7. The needleless syringe of claim 1, further comprising a flange portion that protrudes in a radial direction and is formed on an outer circumferential surface of the piston and a blocker that is provided between the piston and the cylinder and limits a forward movement distance of the piston due to the flange portion blocked when the piston moves forward,
wherein the blocker comprises:
a fixed blocker that is fixedly installed to an inner circumferential surface of the cylinder and has a female thread formed on an inner circumferential surface of the fixed blocker and formed in a ring shape; and
a length adjustment blocker that is screwed to an inner circumferential surface of the fixed blocker, protrudes backward so that the flange portion is blocked by the length adjustment blocker, adjusts a coupling length at which the length adjustment blocker is coupled to the fixed blocker.

8. The needleless syringe of claim 4, further comprising a flange portion that protrudes in a radial direction and is formed on an outer circumferential surface of the piston and a blocker that is provided between the piston and the cylinder and limits a forward movement distance of the piston due to the flange portion blocked when the piston moves forward,
wherein the blocker comprises:
a length adjustment blocker that adjusts a length at which the length adjustment blocker protrudes backward.

9. The needleless syringe of claim 1, wherein a drug supply hole for supplying the drug from the outside by a pressure difference generated when the piston moves backward, is formed in the drug accommodating portion.

10. The needleless syringe of claim 4, wherein a drug supply hole for supplying the drug from the outside by a pressure difference generated when the piston moves backward, is formed in the drug accommodating portion.

11. The needleless syringe of claim 1, wherein the nozzle portion opening/closing valve opens the passage hole with a fluidic pressure applied by the drug from the drug accommodating portion when the piston moves forward, and closes the passage holes when the fluidic pressure is released.

12. The needleless syringe of claim 1, wherein the current supply unit supplies the current supplied from an external power supply source to the first coil and the second coil.

13. The needleless syringe of claim 4, wherein the current supply unit supplies the current supplied from an external power supply source to the first coil and the second coil.

14. The needleless syringe of claim 1, further comprising a piston cover that is provided inside the cylinder to cover an end portion of the piston.

15. The needleless syringe of claim 4, further comprising a piston cover that is provided inside the cylinder to cover an end portion of the piston.

16. The needleless syringe of claim 1, further comprising an elastic member that is installed between the piston and the cylinder and applies an elastic force in a backward movement direction to the piston when the moving magnetic body moves backward.

17. A needleless syringe comprising:
a body;
a solenoid coil wound on an outer circumferential surface of the body;
a cylinder coupled to the body to be in communication with an open front surface of the body and comprising a drug accommodating portion in which the drug is accommodated, and a nozzle portion injecting the drug accommodated in the drug accommodating portion forward;
a moving magnetic body that is long inserted into the body in a longitudinal direction and moves forward by a first magnetic force generated when a current is applied to the solenoid coil;
a stationary magnet that is inserted behind the moving magnetic body inside the body and has a second magnetic force to pull the moving magnetic body when supply of current to the solenoid coil is cut off, to move the moving magnetic body backward;
a piston that is inserted in front of the moving magnetic body inside the body and pressurizes the drug in the drug accommodating portion by an impulse applied by forward movement of the moving magnetic body when the moving magnetic body moves forward;
a nozzle portion opening/closing valve that is provided to open/close a passage hole between the nozzle portion and the drug accommodating portion; and
a forward/backward driving unit that repeats the supply and cut-off of current to the solenoid coil at a preset period, thereby repeating the forward movement and the backward movement of the piston,
wherein the forward/backward driving unit comprises:
a current supply unit that repeatedly supplies a current to the solenoid coil to generate the first magnetic force greater than the second magnetic force during forward movement of the piston and repeatedly cuts off supply of current to the solenoid coil during backward movement of the piston.

18. A needleless syringe comprising:
a body;
a solenoid coil wound on an outer circumferential surface of the body;
a cylinder coupled to the body to be in communication with an open front surface of the body and comprising a drug accommodating portion in which the drug is accommodated, and a nozzle portion injecting the drug accommodated in the drug accommodating portion forward;
a moving magnetic body that is long inserted into the body in a longitudinal direction and moves forward by a first magnetic force generated when a current in a preset first direction is applied to the solenoid coil;
a stationary magnet that is inserted behind the moving magnetic body inside the body and has a second magnetic force to pull the moving magnetic body when supply of current to the solenoid coil is cut off, to move the moving magnetic body backward;
a piston that is inserted in front of the moving magnetic body inside the body and pressurizes the drug in the drug accommodating portion by an impulse applied by forward movement of the moving magnetic body when the moving magnetic body moves forward;
a nozzle portion opening/closing valve that is provided to open/close a passage hole between the nozzle portion and the drug accommodating portion; and
a forward/backward driving unit that repeats the supply and cut-off of current to the solenoid coil at a preset period, thereby repeating the forward movement and the backward movement of the piston,
wherein the forward/backward driving unit comprises:
a current supply unit that repeatedly supplies the current in the first direction to the solenoid coil to generate the first magnetic force greater than the second magnetic force during forward movement of the piston and repeatedly supplies a current in a second direction opposite to the first direction to the solenoid coil during backward movement of the piston.

19. The needleless syringe of claim 17, further comprising a cooling chamber that is provided to surround an outside of the solenoid coil on an outside of the body and absorbs and cools heat generated in the solenoid coil through a cooling fluid.

20. The needleless syringe of claim 17, further comprising a piston cover that is provided inside the cylinder to cover an end portion of the piston.
